(19) United States
(12) Patent Application Publication
(10) Pub. No.: US 2005/0203371 A1
(43) Pub. Date: Sep. 15, 2005

(54) Catheter device

(76) Inventor: Martin Kleen, Neunkirchen (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/040,432

(22) Filed: Jan. 21, 2005

(30) Foreign Application Priority Data
Jan. 21, 2004 (DE) 10 2004 003 082.0

(51) Int. Cl. A61B 5/05

(52) U.S. Cl. 600/407; 600/424

(57) ABSTRACT

The invention relates to a catheter device, comprising a catheter for insertion into a hollow organ, in particular a blood vessel, there being provided inside the catheter a plurality of tube- or balloon-type flexural elements (12, 12a, . . . , 121) which can be filled separately with a filling medium and which are arranged such that they are distributed round the longitu dinal axis of the catheter and at least along part of the length of the catheter, which elements are flexible in a non-pressurized state and stiffen following the build-up of pressure inside and assume a predetermined curved shape, likewise a feed device (5) for the filling medium, which device is connectable to the catheter (2), and which is designed for separate activation of the plurality of flexural elements (12, 12a, . . . , 121) that are provided on the catheter.

### CROSS REFERENCE TO RELATED APPLICATIONS

This application claims priority to the German application No. 10 2004 003 082.0, filed Jan. 21, 2004 which is incorporated by reference herein in its entirety.

### FIELD OF INVENTION

The invention relates to a catheter device, comprising a catheter for insertion into a hollow organ, in particular a blood vessel.

### BACKGROUND OF INVENTION

Flexible catheters which are pushed forward through arteries or veins are used for intravascular or intracardial treatment. At the tip or edge thereof are functional mechanisms, for example to stimulate or cauterize tissue or conduct electrical signals. In order to be able to place the catheter in the correct positions in the cardiac or vascular system, catheters have to be moved and guided by the physician. Such guidance has to be precise, fast and highly flexible, in particular because the vascular system is a convoluted system that contains a number of bends. During treatment what takes up most of the time is the navigation of the catheter. Such catheters are conventionally guided only by manipulating the end that is projecting out of the patient. Turning the catheter, pushing it forward and pulling it back, monitored under X-ray control, combined with the presence of a curvature in the catheter tip makes it possible for the catheter tip to take the desired route, which the rest of the catheter then follows. Such a catheter moved by the user has to be reasonably stiff so that movements can also be further directed to continue round bends. This runs counter to safety requirements however, since a stiff catheter is more likely to cause injuries.

### SUMMARY OF INVENTION

A known guidance method is the use of pull wires that allow the end of the catheter to be moved. The disadvantage thereof is firstly the complexity of the catheter and secondly, the fact that the angle of curvature is limited. A further known technique is magnetic navigation. At the end, which is designed to be very flexible, such a catheter has a tip comprising magnetic material. By applying an external homogeneous magnetic field through the patient, it is possible for the catheter tip and thus the whole of the end of the catheter to align itself along the lines of the magnetic field. By also pushing the catheter forward, it is possible to navigate through complex vascular systems. The above method however requires a substantial outlay in terms of technology, equipment and cost, and furthermore the size of the area that can be navigated is restricted by the dimensions of the magnetic field.

The invention addresses the problem of providing a catheter that allows simple navigation.

To solve the above problem, a catheter device comprising a catheter is provided, inside which are provided a plurality of tube- or balloon-type flexural elements arranged round the longitudinal axis of the catheter and distributed over at least one part of the length of the catheter, which elements can be filled separately with a filling medium, are flexible in a non-pressurized state and which stiffen and assume a predetermined flexural shape when pressure builds up inside them. A feed device for the filling medium is likewise provided, which device can be connected to the catheter and which is designed for separate activation of the plurality of flexural elements that are provided in the catheter.

Using the flexural elements in the catheter tip area and the area adjacent thereto, a curvature of the catheter can be achieved in a simple manner when required. Said curvature allows adaptation to the course of the blood vessel, both at the tip and along the adjacent section of the catheter wherein the flexural elements are incorporated, and for movement of the catheter in a simple manner into a branch of a blood vessel, for example, or if required, it allows the assumption of a particular shape that essentially corresponds to the actual course of the blood vessel. For this purpose, only one or a plurality of flexible flexural elements has or have to be filled in a non-pressurized state with the filling medium, a process which is effected in an automatically controlled manner via the feed device. The rise in pressure leads to stiffening of the flexural element or elements, which assume a predetermined curved shape. According to the filling level, any intermediate shape ranging from completely flexible to completely stiff can be set, there being various degrees of rigidity.

The flexural elements are arranged round the longitudinal axis of the catheter across part of the length of the catheter, the directions of the deformation thereof being usefully aligned in different ways. The flexural elements thus all curve in a different direction with respect to the radial alignment round the longitudinal axis of the catheter with the result that a high degree of local flexibility can be achieved. The aforementioned flexibility is maintained by the arrangement of the flexural elements such that they are distributed in a longitudinal direction over the entire length of catheter along which the flexural elements are arranged. It is thus possible to establish a corresponding curve at virtually any catheter positions.

Here the flexural elements can be arranged in a common longitudinal curve position with respect to the longitudinal axis of the catheter, that is, with respect to the length of the catheter, they are staggered radially outward in a segment-like arrangement at a plurality of points. Alternatively or additionally, it is also possible for said elements to be arranged over a part of the length of the catheter, that is for any number of flexural elements to be arranged so that they are apart and staggered with respect to one another over a particular length of the catheter, in order to achieve adequate curvature options on said longitudinal section a t a plurality of different locations. The length along which the flexural elements are disposed in the aforementioned arrangements can be selected at random, and usually depends on the purpose for which the catheter is used and the possibility of incorporating the flexural elements.

A flexural element itself is usefully made from a non-elastic material. In order to achieve the desired shape in a filled state, the side is non-symmetrical in design, that is the lengths of the side walls are different, with the result that, in the filled state, a curved shape is formed. Usefully, polyurethane or polytetrafluoroethylene are utilized for this purpose, that is, materials that are non-elastic, with the result that any stretching of the material resulting in a more spherical shape is avoided. The filling media can be liquids such as water or saline solution or another, preferably biocompatible fluid, or gaseous media such as air or oxygen or a different, preferably biocompatible, gas.

In order to achieve targeted curvature of the catheter tip, a development of the inventive concept makes provision for the feed device to be designed to allow automatic activation of the required flexural element or elements as a function of at least one item of information relating to the desired direction of curvature of the catheter. An input device for the user to input information relating hereto is usefully provided for this purpose. This can be a monitor for the preferable three-dimensional display of the blood vessel surrounding the catheter, in which display the user can define the direction of curvature by means of a mark or suchlike. This enables the physician to cause the catheter tip to bend in any direction, for example; he merely has to set the corresponding direction of curvature on the monitor within the image display using the cursor or suchlike. The image display shows, preferably in three-dimensional form, a vascular bundle in the immediate vicinity of the catheter, for example. The display can be produced in any manner, via parallel X-ray monitoring or using other sets of image data that were obtained from previous investigations (for example magnetic resonance tomography or computer tomography) and which incorporate the catheter image as captured via the X-ray monitoring. The physician is able to maneuver accordingly within this image to define the direction of curvature.

A particularly advantageous development of the inventive concept makes provision for the feed device to be designed to allow automatic change in the activation of the flexural elements so that there is an essentially locally stable maintenance of the catheter curvature with respect to the hollow organ, said curvature being achieved by activation of one or a plurality of flexural elements when the catheter is moved. This development of the invention makes it possible to, as it were, “freeze” a shape that the catheter has assumed and maintain said fixed shape, even if the catheter is further inserted or withdrawn. To a certain extent, the curvature progresses along the catheter while it is being moved, yet it remains fixed with respect to the position in the blood vessel. This has the considerable advantage that, during the movement of the catheter, a largely optimum adaptation of the shape of the catheter to the actual curvature of the vascular system can be achieved, as a result whereof there is less risk of injury and irritations of the vascular wall can be reduced. As a result of reduced friction on the vascular walls, the risk of injury from abrasion of the vascular endothelium is likewise reduced. This applies to both the insertion and the removal of the catheter, which can be effected more quickly and more safely since entanglement of the catheter in internal vascular structures can largely be avoided as a result of the adaptation of the shape. Furthermore, optimum use can be made of the force exerted by the physician or by an automatic insertion system as the catheter is guided forward, if the catheter is fixed in shape, that is stiff, at least locally. The result thereof is that unintentional prolapse is less probable. Less force is needed to push the catheter forward since the reduction in the friction created by the shape-adapted catheter on the vascular walls means that less force goes to waste.

In order for the shape-adaptation to be carried out continually even when the catheter is being moved, provision is usefully made during a change in position of the catheter for automatic activation of the flexural elements that are required to maintain and fix the curvature that has been set. In order for this to be achieved in an optimal manner in the feed device, said feed device is designed to be activated automatically, on the basis of information that has been input by the user with respect to the local curvature of the catheter tip that is required and of information relating to the movement of the catheter. After the catheter tip, which is first to negotiate bends in the vascular system, has been bent accordingly for simple navigation, information is consequently available relating to the shape that the catheter needs to adopt locally with respect to the vascular system. On the basis of the above information, and in conjunction with information relating to the movement of the catheter, it is now possible to retain the local shape of the blood vessel when moving the catheter to insert it into the blood vessel; it is merely necessary to activate the corresponding flexural elements in the subsequent sections of the catheter that are negotiating the bend in the blood vessel. This means that pushing the catheter forward x cm results in the curvature being shifted or moved on x cm toward the outer end of the catheter. A corresponding situation also applies of course to the withdrawal of the catheter, and here, too, a movement of x cm similarly means that the curvature is shifted the same distance toward the catheter tip.

In order to record movement information, it is conceivable for one or a plurality of position sensors to be provided on the catheter, the position of said sensors being determined using a position detection system in which information relating to the movement of the catheter can be determined via the position sensors. Such position detection systems are quite well known, and are usually based on electromagnetic signals that can be detected by the position detection system working in conjunction with the position sensors. As a result it is possible to determine by precisely what length and in which direction the catheter is being moved.

Alternatively there is the option of moving the catheter using a mechanical pushing device that provides the information relating to the movement of the catheter. Such automatic catheter insertion systems are known; they are usually controlled by stepping motors and allow the catheter to be moved by precisely definable lengths.

As disclosed above, the user usefully has the option of defining, via the input device, the desired curvature of the catheter, irrespective of whether it is at the tip, or at a position along the catheter. He can likewise select the function for maintaining the curvature via the input device. In other words, the physician can, as it were, “freeze” the shape of the catheter where curvatures that have already been set are available. The shape of the catheter tip can, of course, always be altered during further insertion of the catheter, for it is fundamentally possible during the further insertion movement for one or another curvature in the blood vessel to be traversed. Additional curvature information is of course subsequently taken into account in the context of the maintenance of the curvature.

Finally, it is also conceivable for local deactivation of the maintenance of the curvature to be possible. In other words, the user has the opt ion despite having selected the “freeze” function, of locally deactivating said function if this is necessary for any reason. Local deactivation has the effect that the flexural elements located at and subsequently moved to one or a plurality of locally detectable flexural positions are no longer activated accordingly. Said function can likewise be activated again, however, after the original flexural parameters are once again known to the feed device.

### BRIEF DESCRIPTION OF THE DRAWINGS

Further advantages, features and details of the invention will become apparent from the embodiment described below and from the drawings. The drawings show:

FIG. 1 a diagram showing the principle of a catheter device according to the invention,
FIG. 2 a diagram showing the principle of a flexural element in a non-pressurized state,

FIG. 3 a diagram showing the flexural element from FIG. 2 in a filled state,

FIG. 4 a diagram showing the catheter tip having two flexural elements acting in the opposite direction,

FIG. 5 a cross-sectional view through a catheter having a plurality of flexural elements staggered radially outward and arranged so that they are spread out,

FIG. 6 a diagram showing the catheter course achieved by activating various flexural elements, and

FIG. 7, 8 and 9 diagrams demonstrating the principle of the “freeze function” used in order to maintain a set shape of the catheter despite further movement of the catheter in the blood vessel.

### DETAILED DESCRIPTION OF INVENTION

FIG. 1 shows a catheter device 1 according to the invention comprising a catheter 2, at the free end of which that is not to be inserted into the patient a connection device 3 is provided, which is connected to a connection device 4 that forms part of a feed device 5 for a liquid or gaseous filling medium. Using said feed device 5, a liquid or gaseous filling medium can be supplied to the individual flexural elements that are incorporated in the catheter and which are hereafter described in further detail. The feed device 5 is connected t o an input device 6 comprising a monitor 7, a keyboard 8 and a mouse 9. By means of said device, the user, observing an image on the monitor 7, supplied for example by an X-ray image taken in parallel by an X-ray device 10 during the invasive procedure, or optionally using an image data set 11, obtained for example by magnetic resonance tomography or computer tomography, can input the direction in which the catheter is to be bent.

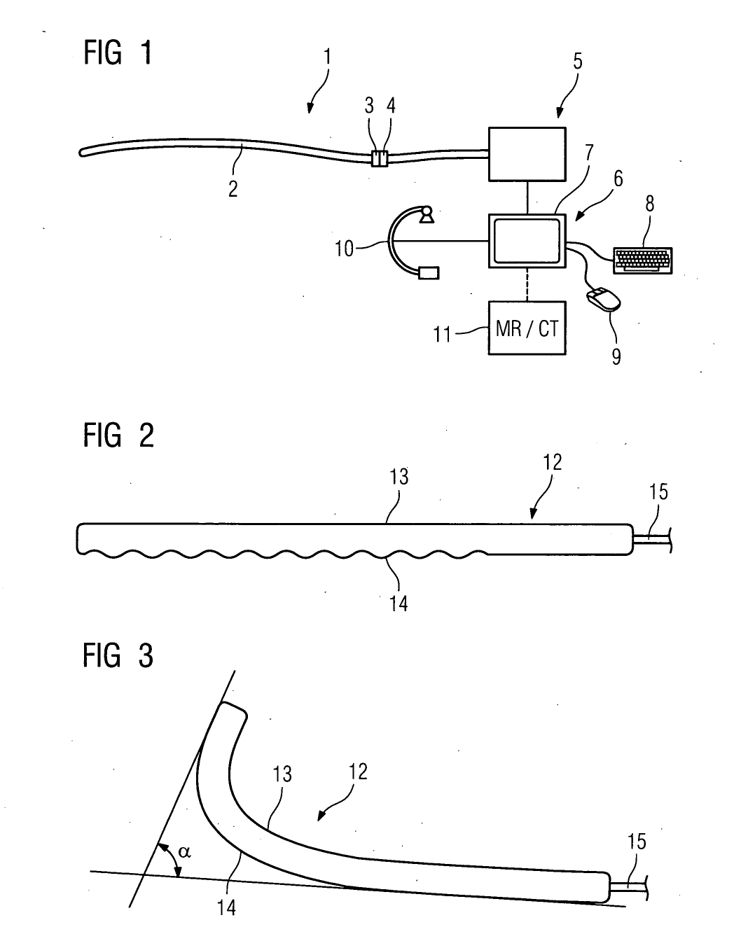

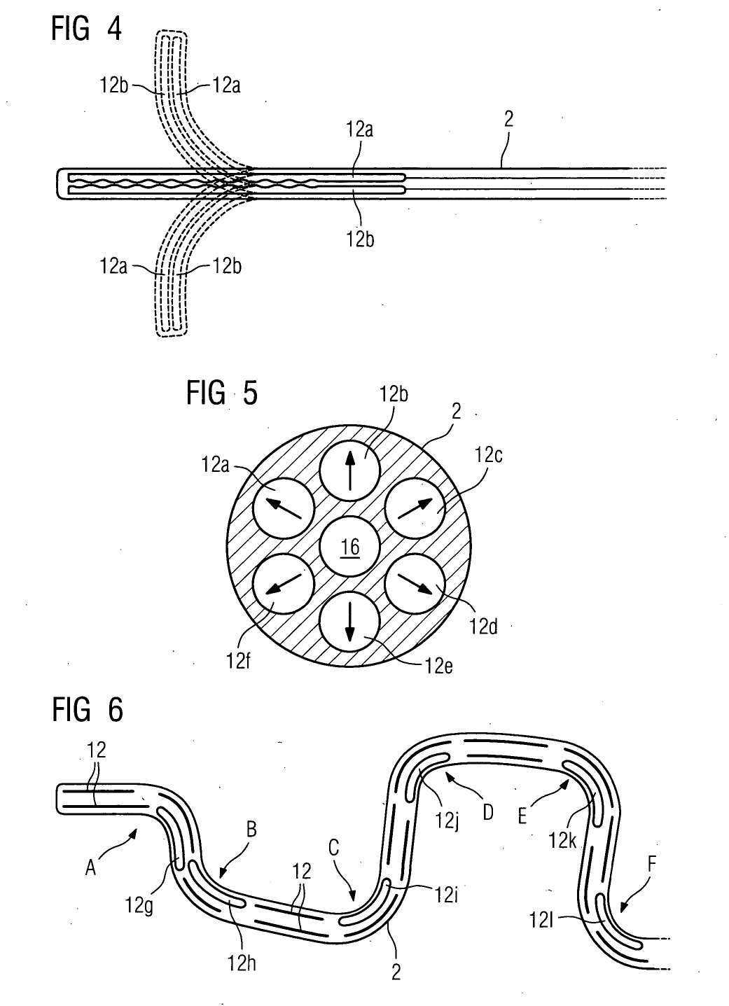

The functional principle is that there are incorporated in the catheter one or a plurality of flexural elements which can be directed into a particular shape when the pressure inside builds up. FIG. 2 shows a diagram illustrating the principle of a flexural element 12, which is tube-like in design. Said element consists of a non-elastic plastic material, for example PUR or PTFE, but any other plastic can be used. The flexural element 12 has on one face a shorter side segment 13, and on the opposite face a longer wall segment 14 is provided, in other words, the side is non-symmetrical overall. If filling medium, for example, water, saline solution or air or oxygen is now supplied via the feed line 15, then the pressure builds up inside, resulting in the flexural element 12 having a maximum volume while covering a minimum surface. Since the side is non-elastic, no stretching can occur. The side 14 stretches such that the flexural element 12 assumes the shape shown in FIG. 3, in which it is sufficiently stiff because of the pressure inside. The diagram shows how a curvature that is created by the geometrical shape of the flexural element 12 can be set in this way. In the example shown, the angle of curvature a is drawn. If such a flexural element is now incorporated into the catheter 2, as shown in FIG. 4 in a diagram showing the principle involved, a defined deformation of the catheter can be achieved. In a non-pressurized state the flexural element 12 is flexible, that is, it has not been stiffened and the shape thereof is determined by the shape of the catheter or catheter casing. The catheter casing consists, for example, of a slightly elastic plastic material and has sufficient stiffness or rigidity to allow manipulation of the catheter. The diagram shows how the shape of the catheter 2 changes when such a flexural element is subject ed to pressure, as shown by the dotted lines in FIG. 4. The catheter bends upward or downward for example as shown in the diagram showing the principle involved in FIG. 4 and essentially has a flexure of 90°, effected by the defined alteration in the shape of the flexural elements. If pressure on a flexural element is relieved again, said element becomes flexible again and collapses as it were, optionally assisted by the restoring force of the slightly elastic catheter casing.

FIG. 4 shows the catheter 2, incorporating two flexural elements 12a, 12b that are essentially identical in design, both therefore having a short and a longer side face. Depending on which flexural element is filled, the direction of curvature changes, once said two elements have exhibited different preferential directions of curvature. If flexural element 12a is filled, then the catheter tip curves upward as shown in FIG. 4, and the flexible, non-pressurized flexural element 12b automatically assumes the same curvature. Conversely, i f flexural element 12b is filled, the catheter tip curves downward because of the preferential direction of said element as shown in FIG. 4, and here the non-pressurized flexural element 12a assumes the same change in shape. The respective radius or angle of curvature a that can be achieved depends on the ratio for the material length in the side sections, which face each other and are of different lengths. According to the design and dimensions thereof, the angle of curvature can consequently be varied, and likewise, of course, the position of the point of flexure, that is, depending on where the segment of the side that is “long” in terms of the material used is provided in relation to the length of the flexural element.

FIG. 5 shows a cross-sectional view of a catheter 2, around the central aperture of which 16, in which, for example, a further working catheter is guided or signal or control lines and so on, six flexural elements 12a, 12b, 12c, 12d, 12e and 12f are arranged, radially staggered outward and in the example shown distributed symmetrically. Each of the flexural elements 12a-f can be controlled separately via a separate feed line that is not shown in any further detail. The positioning, alignment and design of the flexural elements in the above arrangement is such that each flexural element has its own preferential direction of curvature, said preferential direction of curvature being directed in a different direction in each case. Said preferential directions of curvature are shown by the respective arrows in the flexural elements. The arrow shows how the respective flexural element—as shown for example in FIG. 4—starts off from a virtually straight catheter shape and then bends in the direction of the arrow. If a plurality of flexural elements are therefore incorporated in the catheter and the directions of the deformation of the individual flexural elements are directed as shown in FIG. 5, then a different direction of flexure can be achieved by increasing the pressure in each separate flexural element. Combinations are also possible of course; in other words, pressure can be applied to two adjacent flexural elements, such that the resulting direction of flexure is the direction that lies between the individual main element-related directions. It is also conceivable of course for pressure to be applied to all the flexural elements so that the individual effects thereof are cancelled out, but the catheter itself stiffens considerably in the zone where the flexural elements have been provided. With respect to the longitudinal axis of the catheter, the flexural elements are staggered radially outward and can either be positioned in segments, that is, a plurality of flexural elements are arranged at the same longitudinal position, or alternatively it is also conceivable for the flexural elements to be positioned such that they are staggered outward but also overlap one another, in other words in the style of a spiral-shaped arrangement. Since each flexural element can also be activated separately here, a locally defined curvature can also be achieved here.

FIG. 6 is a diagram showing the principle involved in an example of the deformation of a catheter 2 that is achievable by separate activation of individual flexural elements. A plurality of individual flexural elements 12 are distributed along the length of part of the catheter. Said elements can either be distributed in segments (flexural elements staggered radially outward at a plurality of defined longitudinal positions) or staggered in a spiral arrangement (flexural elements staggered radially outward but also overlapping one another at the respective different longitudinal positions). A total of six different flexural points A, B, C, D, E and F are shown in FIG. 9. In order to achieve flexure at flexural point A, the flexural element 12g is activated and the adjacent, in particular the facing flexural elements 12 remain non-pressurized and therefore flexible. In order to achieve flexure at flexural point B, flexural element 12h is activated, and in order to achieve flexure at flexural point C, flexural element 12i is activated. In order to achieve flexure at flexural point D, a similar procedure is used and here the flexural element 12j is activated by the feed device. In order to achieve flexure at flexural point E, flexural element 12k is activated and finally in to achieve flexure round flexural point F, flexural element 12l is activated. The diagram shows how the circumstance that each of said flexural elements has a defined preferential direction of curvature and assumes said curvature when in a pressurized state results in the whole of the catheter in the respective area assuming the corresponding curvature and consequently the shape shown in FIG. 6, which is highly convoluted, appears.

The flexural elements can be of any length and to allow sufficient flexure with respect to the diameter of the catheter, they should be at least 1 cm or more in length. The diameter thereof varies according to the type and diameter of the catheter and the type of arrangement of the flexural elements and the number thereof. It should be at least 1 mm or more in length.

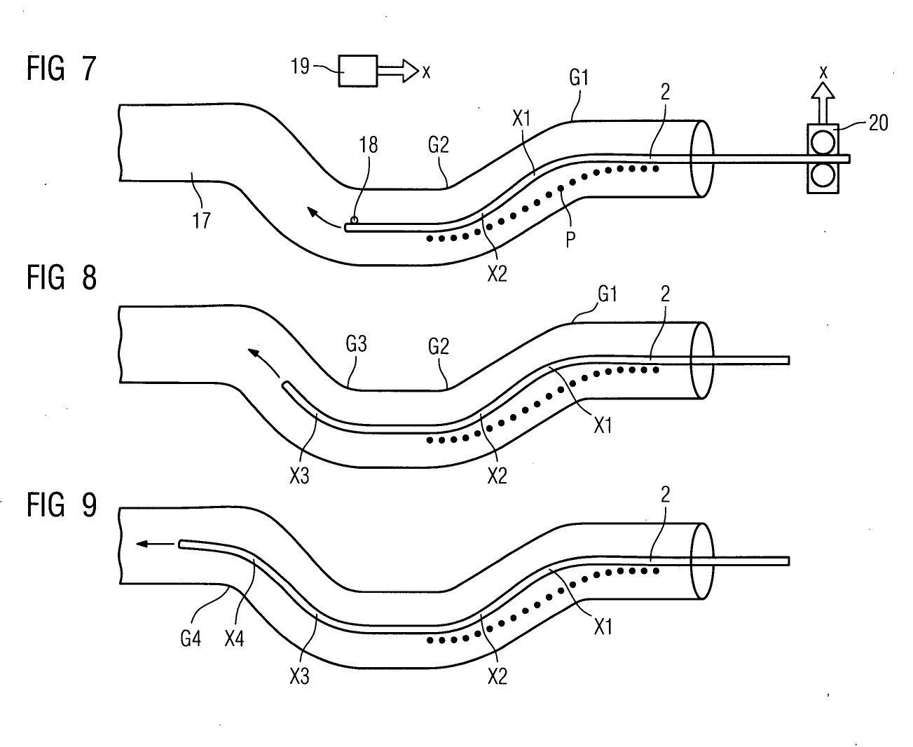

FIGS. 7, 8 and 9 show the “freeze function”, which the user can select via the input device 6. The aforementioned function allows a set shape of the catheter 2 or local curvature of the catheter to be maintained with respect to the position in the blood vessel. As shown in FIG. 7, the catheter 2 in the blood vessel 17 follows a curve in zone X1 on the curve G1 in the blood vessel and a second curve in zone X2 on curve G2 in the blood vessel, thus corresponding to the course of the blood vessel. As is indicated by the dots P, this results in a defined curvature of the catheter. If the catheter is now further inserted into the blood vessel following the direction of the arrow, the catheter reaches the curve G3 in the blood vessel. The physician, who is familiar with this curvature from X-ray images for example, defines on the monitor 7 of the input device 6 what curvature of the catheter should be effected, and the feed device 5 activates the corresponding flexural elements required. The catheter follows a curvature X3 during the forward movement. At the same time, however, the diagram shows how catheter curvatures X1 and X2 for the respective local curves G1 and G2 in the blood vessel remain fixed, in other words, the curvature of the catheter moves along the catheter as it were.

A simple procedure is used if the catheter is moved further on, as shown in FIG. 9. It reaches the zone of curve G4 in the blood vessel. The physician again defines on the monitor 7 the curvature that is to be effected in the catheter tip, which curvature is then activated, such that the catheter again becomes curved as at X4. The curvatures X1, X2 and likewise the curvature X3 that was defined shortly beforehand are maintained. In order to effect the above continuous adaptation or maintaining of shape, it is firstly essential to know what is the type of curve that is to be maintained, said information being known from the definition of the curvature of the tip that is automatically effected beforehand and carried out by the user since the catheter tip is the part of the catheter that is first to negotiate a curve in the blood vessel. Furthermore, it is necessary to determine the movement of the catheter through the blood vessel. For this purpose, as shown in FIG. 7 by way of example, a position sensor 18 can be arranged on the tip of the catheter, which sensor interacts with an external position detection system 19 through which information relating to the distance moved x can be determined. Alternatively there is also the option here of determining said information x via an automatic pushing device 20, which records the automatic movement of the catheter. All the information required is supplied to the feed device 5, which comprises an appropriate control device to process the data and activate the corresponding flexural elements required.

1.-10. (canceled)

11. A catheter device, comprising:
a catheter for inserting into a hollow organ;
a plurality of hollow flexural elements arranged within the catheter around a catheter longitudinal axis and distributed along a catheter length; and
a supply device connected to the catheter for feeding a filling medium to the flexural elements, wherein
the flexural elements are flexible in a non-pressurized state and adapted to be filled with the filling medium,
the filling medium is adapted to apply an internal pressure to the flexural elements causing the flexural elements to stiffen and assume a curved shape, and
the supply device is adapted to individually activate at least one single flexural element by selectively feeding the filling medium to the single flexural element.

12. The catheter device according to claim 11, wherein the supply device is adapted to individually activate every single flexural element by selectively feeding the filling medium to each flexural elements so that the internal pressure within every single flexural element is controllable using the supply device.

13. The catheter device according to claim 11, wherein the flexural elements are shaped as tubes or as balloons.

14. The catheter device according to claim 11, wherein the hollow organ is a blood vessel.

15. The catheter device according to claim 11, wherein the supply device is further adapted to cause a curvature of a catheter tip by selectively feeding the filling medium to such flexural elements of the plurality of flexural elements which are arranged adjacent the catheter tip and constructed to achieve the curvature when supplied with the filling medium.

16. The catheter device according to claim 15, wherein the supply device activates the flexural elements upon information data related to a desired curvature and fed to the supply device.

17. The catheter device according to claim 16, further comprising an input device for inputting the information data.

18. The catheter device according to claim 17, wherein the input device comprises a monitor device for displaying the hollow organ and the inserted catheter, the monitor device adapted to receive the information data via a marking set by a user on the monitor.

19. The catheter device according to claim 11, wherein the supply device is further adapted to feed the filling medium to the flexural elements during operation of the catheter device such that a desired curvature of a catheter tip caused by stiffened flexural elements is maintained relative to the hollow organ during a movement of the catheter.

20. The catheter device according to claim 19, wherein the supply device is arranged and constructed to be activated by an information input by a user, the information including curvature data related to the curvature of the catheter tip and/or movement data related to the movement of the catheter.

21. The catheter device according to claim 11, wherein the supply device is arranged and constructed to be activated by an information input by a user, the information including curvature data related to a curvature of a catheter tip and/or movement data related to a movement of the catheter.

22. The catheter device according to claim 21, wherein
at least one position sensor is arranged on the catheter for determining a position of the inserted catheter using a position detection system, and
the movement data of the catheter are determined by the position detection system using a sensor signal of the position sensor.

23. The catheter device according to claim 21, wherein the catheter is moved by a mechanical pushing device, the mechanical pushing device adapted to supply the movement data.

24. The catheter device according to claim 19, wherein a function for maintaining the desired curvature is selectable via an input device by a user.

25. The catheter device according to claim 24, wherein the function for maintaining the curvature can be deactivated at least partially so that at least one flexural element formerly involved in maintaining the desired curvature is selectively deflated with the remaining pressurized flexural elements keeping their internal pressure.
